(19) United States
(12) Patent Application Publication
(10) Pub. No.: US 2022/0175983 A1
(43) Pub. Date: Jun. 9, 2022

(54) Device With A Self-Disinfecting Surface

(71) Applicant: TOUCH UV, INC., Carlsbad, CA (US)

(72) Inventors: Stephen R. Wood, (US); John Hradnansky, (US)

(73) Assignee: TOUCH UV, INC., Carlsbad, CA (US)

(21) Appl. No.: 17/113,637

(22) Filed: Dec. 7, 2020

(51) Int. Cl. A61L 2/10; A61L 2/26

(52) U.S. Cl. CPC A61L 2/10; A61L 2202/14; A61L 2202/11; A61L 2/26

(57) ABSTRACT

Provided herein is a self-contained, self-disinfecting device which automatically disinfects a surface of the device using internally-generated or applied UV (Ultraviolet) light. The device may be a door handle, push plate, light switch, shopping cart, or the like.

Described herein is a device comprising a UV-transparent or UV-translucent article and at least one optically connected UV light source. The UV light source acts to automatically disinfect a surface of the article by transmitting UV light or radiation to the surface.

### DETAILED DESCRIPTION OF THE DRAWINGS

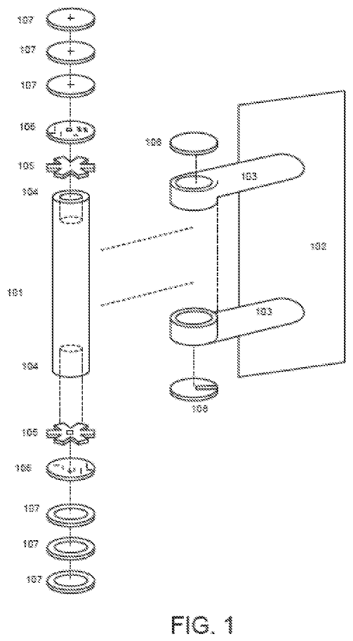

FIG. 1 illustrates an example of a door handle device as described herein. The device includes a UV-transmissive handle which may be secured to a door via a handle mounting bracket and mounting plate. The bracket and/or plate may be constructed of UV-opaque material. The UV light in this device is provided by two UV-emitting LEDs (“UVEDs” or “UV LEDs”) which are powered by batteries.

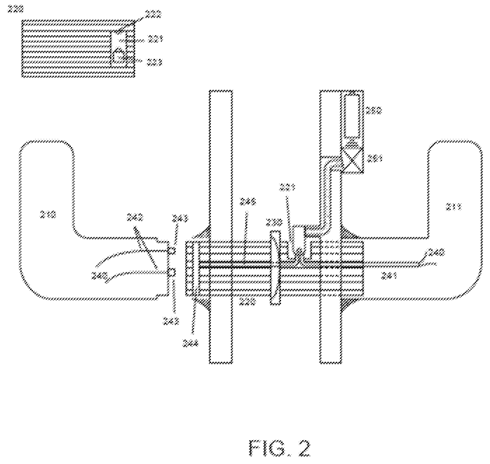

FIG. 2 illustrates a second alternative door handle embodiment which includes a battery power supply and use-activated disinfecting.

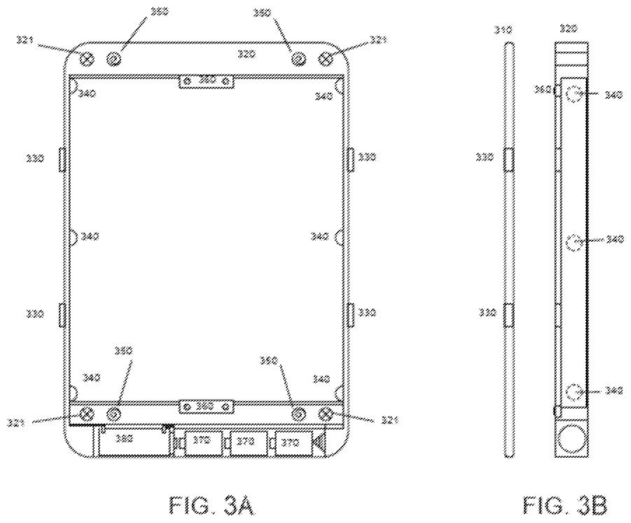

FIG. 3 illustrates a door push-plate with pressure-activated disinfecting surface (FIG. 3A—front view; FIG. 3B—side view).

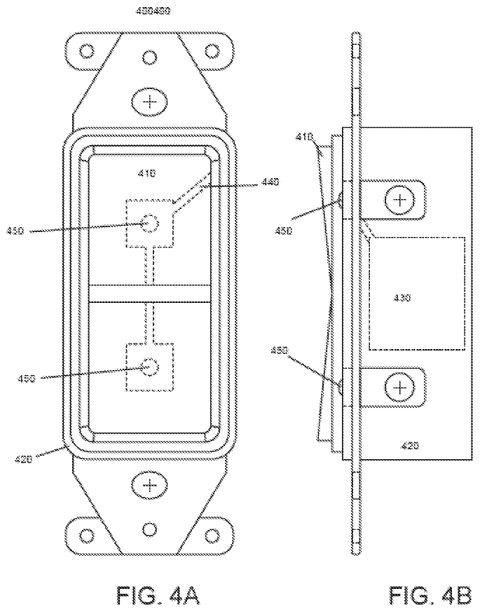

FIG. 4 shows a self-disinfecting light switch embodiment (FIG. 4A—front view; FIG. 4B—side view).

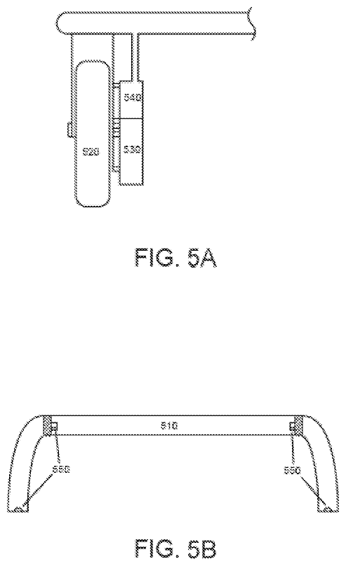

FIG. 5 shows a self-disinfecting grocery cart or other push cart handle. FIG. 5A shows a wheel of the cart with connected accelerometer; FIG. 5B shows the handle with integral UV light sources.

### DETAILED DESCRIPTION

As used herein, an “article” comprises a surface with which humans come in contact that can be manufactured at least in part from UV-translucent or UV-transparent material. Such surfaces include, but are not limited to, door opening means; drawer opening means; power switch means; light switch means, hand/hold rails, push bars, and keypad or touchscreen means; countertops, and toilet seats. Other “articles” may include but are not limited to subway/bus hold bars; staircase railings; balcony railings; point of sale consoles; remote controls; and keyboards.

Door opening means comprise, but are not limited to:
Push handles (vertically or horizontally oriented)
Pull handles (vertically or horizontally oriented)
Push plates (all types)
Rotating door levers
Rotating knobs
Push buttons

Drawer opening means comprise, but are not limited to:
Surface-mounted pull handles (vertically or horizontally oriented)
Surface-mounted knobs
Recessed pull handles
Spring-actuated push plates

Power switch means comprise, but are not limited to:
Toggle switches
Rocker switches
Push buttons

Light switch means comprise, but are not limited to:
Toggle switches
Rocker switches
Push buttons
Touch switches

Touch screen means comprise, but are not limited to:
Point of Sale screens
Personal computer screens
Industrial Controls
Driving controls
Presentation screens
Direction kiosks

The articles described herein comprise a material which is at least translucent to at least part of a spectrum of UV light which is emitted by at least one optically connected UV light source. In an alternative embodiment, the material is transparent to UV light. Suitable UV-transparent or translucent materials for use in the articles described herein include, but are not limited to:
Acrylic
Plexiglas
Glass
Fiberglass
Polyethelene Terephthalate (PET) or other thermoplastic
Quartz glass
Fused Silica
Cyclic Olefin Copolymer (COC) or other advanced polymer

Those skilled in the art will understand that the choice of material will affect the degree or magnitude to which the article will transmit UV light, and the frequency at which it will do so.

The article may further comprise a UV-opaque material which confers structural rigidity or other desirable characteristics to the article. For example, a door handle may comprise a metallic core and a UV-transparent or translucent outer sheath which can transmit UV light to the surface of the article. The UV-opaque material may comprise metal, ceramic, carbon fiber, plastic, or other materials.

In instances in which the article or its surfaces are of an extended or irregular geometry, the waveguide properties of the UV-transparent/translucent material can serve to transmit sufficient UV light for surface disinfection to all parts of the article's surface.

The device described herein also comprises at least one UV light source, which emits light of wavelengths in the ultraviolet spectrum. Suitable wavelengths of UV light for use in the articles described herein will be from about 100 to about 400 nm. Narrower UV spectra may be employed, for example limited to the UV-C band (about 100 to about 280 nm); from about 200 to about 230 nm; or from about 250 to about 270 nm. In one embodiment, the UV light spectrum will include 254 nm. In an alternative embodiment, the UV light spectrum will include 265 nm.

Again, those skilled in the art will understand that trade-offs exist in the choice of UV wavelength or spectra, and that some selections may suit certain applications or articles more than others. For example, it is widely held that the spectra at or about 254 nm, or the spectra at our about 265 nm, are most effective for actual disinfection (depending on the target pathogen), while shorter wavelengths (from about 200 nm to about 230 nm for example) will still disinfect and may be safer to human skin or touch owing to the shorter skin-depth of this wavelength of UV radiation. Other wavelengths and spectra (from about 100 nm to about 200 nm for example), though still capable of disinfecting, are also capable of generating ozone upon contact with oxygen through a process known as photolysis, possibly making this choice of wavelength or spectra less desirable for certain articles depending on their application environment.

Any light source which emits UV light of the desired wavelength and intensity may be employed in the device described herein. Suitable light sources include, but are not limited to, incandescent bulbs; fluorescent bulbs, halogen bulbs; xenon bulbs; mercury-vapor lamps, light-emitting diodes (LEDs), and lasers or other coherent light sources.

The UV light source may be powered externally or may comprise a power source. Suitable power sources include, but are not limited to:
Batteries (all types, including rechargeable)
Inductively coupled power sources (including “Wi-tricity”)
Kinetically generated power
Solar or photovoltaic power

The device may also comprise a visible power indicator light. Such a visible power indicator light may be configured to indicate that the device is properly functioning

The UV light source will operate on a duty cycle which is sufficient to disinfect the surface of the article. Suitable duty cycles include, but are not limited to:
100% at continuous power
Timed pulse, <100% power duty cycle
Use, touch, or motion activated pulse, <100% power duty cycle
Algorithm controlled (e.g., based on day/time or use history) <100% power duty cycle.

One embodiment of the device described herein is shown in FIG. 1. FIG. 1 is a schematic drawing showing a handle 101, which is made of a UV-transmissive material. The handle 101 may be attached to a door, drawer, or any other article that opens when the user grasps and pulls on the handle. The handle 101 may be attached to the door, drawer, etc. directly or via a mounting plate 102 by one or more handle mounting brackets 103. The handle 101 may be straight and cylindrical, as shown in the figure, or any other convenient shape, and may be curved or angled as desired. In the embodiment shown in FIG. 1, the handle 101 includes two ends 104. A UV-light source 105 may be mounted at each end 104 or at both ends. Each UV-light source 105 is driven by a driver circuit 106 and powered by one or more batteries 107. The batteries may be accessed for removal and/or replacement via a battery retainer 108.

A second embodiment of the device described herein is shown in FIG. 2. FIG. 2 is a schematic drawing showing a cross-section of a handle mechanism which will typically be attached to a door. The device may be self-contained within an enclosure or may be mounted directly to a door in which a suitable hollow space to enclose the elements of the device exists or has been created. In FIG. 2, the handle 210 on one side of the door is movable, while the handle 211 on the other side of the door is fixed. Two movable handles 210 may be used together, or two fixed handles 211, as required by the user. Although FIG. 2A shows a handle with a 90-degree bend, a handle as described herein may be of any desired shape which permits the user to operate the handle mechanism. At least one movable handle 210 and the fixed handle 211 should be made of at least UV-translucent material. The handles 210 and/or 211 may be connected via a connecting member 220. In one embodiment the connecting member 220 may be in the form of a spline gear, as shown in FIG. 2, which can be operatively connected to a conventional door latch mechanism directly or via door latch connecting means 230.

Handles 210 and/or 211 may include one or more UV-light source(s) 240. The UV-light source(s) are connected to a power source 250. Power source 250 may be a removable battery pack, a fixedly mounted battery holder, or other self-contained source of direct current that is in close proximity to the door handle and may be part of the device. Alternatively, power source 250 may be a source of alternating current that is part of a building's wiring, in which case a transformer that changes alternating current to direct current may be required.

For a fixed handle 211 a UV-light source 240 may be hard-wired via power wires 241 to the power source 250. For a moveable handle 210, a UV-light source 240 may be operatively connected to the power source 250 via any suitable connecting means which permit the movement of the handle 210 while preserving the electrical connection between the UV-light source 240 and the power source 250. One example of such connecting means, shown in FIG. 2A, includes power wires 242, which connect the UV light source 240 to power transfer posts 243. Power transfer posts 243 make contact with a power contact surface 244 which is operatively connected via second power wires 245 to the power source 250.

In embodiments which include a spline gear, power wires 241, 245 may connect to the power source 250 via an opening 221 which provides access to an on-off contact 222 and a power splitter with a contact switch 223, as shown in the inset in FIG. 2. This on-off contact 223 may be used to initiate a power duty cycle or disinfecting sequence.

If the UV light source(s) 240 are light-emitting diodes (“LEDs”), the device may also include one or more LED drivers 251. In the embodiment shown in FIG. 2A, a single LED driver is incorporated within the power source 250, which is a removable battery pack in this figure. Alternatively, one or more LED drivers may be located at any convenient location within the device which provides for electrical contact between the LEDs and the power source.

A third embodiment of the device described herein is shown in FIG. 3. This embodiment is a schematic view (FIG. 3A—front view; FIG. 3B—side view) of a plate 310 which may be attached to a door, and which activates when the plate 310 is pushed to open the door. While the plate 310 shown in FIG. 3 is approximately rectangular in shape, plate 310 may be formed in any shape which is desired. Plate 310 is made of UV-transparent, translucent, or transmissive material and may be of any suitable thickness.

In the embodiment shown in FIG. 3, plate 310 is mounted to a housing 320 via mounting tabs 330. In its un-activated state, plate 310 will be mounted to housing 320 in any suitable manner such that, when plate 310 is pressed by the user, switch means are activated which result in one or more UV light sources 340 being energized, thereby internally illuminating plate 310 with UV light. The switch means may include one or more springs 350 and one or more electrical switches 360. In its resting state, the spring(s) 350 hold the plate 310 at a functional distance from the housing 320, such that the switch(es) 360 are electrically open. When plate 310 is pressed by the user, the spring(s) 350 compress such that plate 310 makes contact with switch(es) 360, electrically closing the switches. Although FIG. 3 shows an embodiment that includes four coil springs 350 and two switches 360, it will readily be appreciated that the number and configuration of the springs 350 and switches 360 may be easily changed as required. While FIG. 3 shows four mounting tabs 330, any number of tabs, or other means, may be used to mount the plate 310 to the housing 320, as long as they provide for secure mounting of the plate and permit the switch(es) 360 to be activated when plate 310 is pressed.

The embodiment shown in FIG. 3 also includes a self-contained power supply with batteries 370. In the embodiment shown in FIG. 3, six UV-emitting LEDs 340 are shown; accordingly, the power supply also includes an LED driver 380. The LED driver 380 need not be positioned as shown but may be mounted in any position which provides electrical continuity between the power supply and the UV light source. Although the embodiment shown in FIG. 3 includes batteries, as above, the UV light source(s) may be powered via a source of alternating current that is part of a building's wiring, in which case a transformer that changes alternating current to direct current may be required.

The embodiment shown in FIG. 3 is attached to an underlying surface via mounting screws 321. However, any suitable means for securely mounting the device to an underlying surface may be used, such as bolts or nails, and including adhesive means such as glue, epoxy, or tape; alternatively, when the device includes a self-contained power supply, the device may be removably mounted to an underlying surface using, for example, removable adhesive, Velcro®, or other similar means.

A further embodiment is shown in FIG. 4 (FIG. 4A—Front view; FIG. 4B—Side view). This embodiment is switch 400, which may be a light switch. Switch 400 includes a plate 410 for activating and deactivating the switch, and a housing 420 in which circuitry 430 is enclosed. Plate 410 will be made of UV-transmissive material. When switch 400 is activated by the user, one or more UV light sources 450 are energized. Power to energize the UV light source(s) 450 will typically be drawn from the switched circuit via at least one power wire 440. The UV light may remain energized when switch 400 is in the “on” position; alternatively, circuitry 430 may also include a timer which will deactivate the UV light source(s) after a desired period of time, which may be pre-set at the factory, and/or adjusted by the end user, as desired. For improved safety, it may be desirable for the switched circuit, upon activation, to initially pause for a predetermined length of time, then activate a disinfecting cycle, and subsequently deactivate or de-energize.

The embodiment shown in FIG. 5 is a self-disinfecting handle 510 for a shopping cart. Handle 510 is made from UV-transmissive material(s). As shown in FIG. 5B the handle 510 may incorporate one or more UV light sources 550. In one embodiment the UV light source(s) may be LEDs. The cart includes wheels 520. The cart may also include a power source 530 which may include at least one battery. One or more of the wheels 520 may be attached to the power source 530 via a power generator unit that will charge the at least one battery when the wheel 520 turns as the cart is pushed (FIG. 5A). Alternatively, the cart may be provided with means for attachment of the cart to external battery charging means, either via a cord and plug, or electrical contacts which will permit the battery to be charged when the cart is placed into a docking station or other receptacle, or the cart might simply use replaceable battery power sources. When the UV light sources are LEDs, one or more of the one or more power source(s) 530 may also incorporate an LED driver 540. Depending on the number of LEDs used, and whether the power source(s) are wired in series or parallel, more than one LED driver 540 may be required. The power source 530 may also include means for detecting when the cart is moving, such as an accelerometer or equivalent circuit, and a timer that will activate the UV light source(s) 550 for a predetermined length of time. The timer may activate the UV light source(s) 550 at a fixed amount of time after the power source 520 senses no motion from the wheels 520. Alternatively, the timing system will be activated once it senses initial motion from the wheels 520. In the embodiment shown in FIG. 5, the handle 510 includes LEDs at each end of the handle pointing towards each other. This particular embodiment also includes one additional LED at each end of the 90-degree turn in the handle each pointing towards the 90-degree turn. This configuration is merely exemplary; the skilled person will be able to position the UV light sources at different places within the handle, and also around the basket area to cover any surface subject to contamination, as desired and/or required.

What is claimed is:

1. A device comprising a UV-transparent or translucent article comprising a surface configured for human contact, and an optically connected UV light source.

2. The device of claim 1, the article selected from the group consisting of door opening means, drawer-opening means, power switch means, light switch means, keypad or touchscreen means, hold- or hand-rail means, and push bar means.

3. The device of claim 2, wherein the door opening means comprises a push handle, a pull handle, a push plate, a rotating or fixed door lever, or a rotating or fixed knob.

4. The device of claim 2, wherein the drawer opening means comprises a surface-mounted pull handle, a surface-mounted knob, a recessed pull handle, or a spring-actuated push plate.

5. The device of claim 2, wherein the power switch means comprises a toggle switch, rocker switch, push button, or touch switch.

6. The device of claim 2, wherein the light switch means comprises a toggle switch, rocker switch, o push button, or touch switch.

7. The device of claim 1, wherein the article is transparent or translucent to UV light.

8. The device of claim 7, wherein the article comprises a UV-transparent or translucent material selected from the group consisting of acrylic, Plexiglas, glass, fiberglass, and PET or other thermoplastic, quartz glass, fused silica, COC or other advanced polymer.

9. The device of claim 1, wherein the UV light source emits a spectrum of light comprising wavelengths from about 100 to about 400 nm.

10. The device of claim 9, wherein the UV light source emits a spectrum of light comprising light wavelengths in the UV-C band.

11. The device of claim 10, wherein the UV light source emits a spectrum of light comprising light wavelengths from about 250 to about 270 nm.

12. The device of claim 11, wherein the UV light source emits UV light comprising light with a wavelength of about 254 nm or about 265 nm.

13. The device of claim 10, wherein the UV light source emits a spectrum of light comprising light wavelengths from about 200 to about 230 nm.

14. The device of claim 1, wherein the UV light source comprises an incandescent bulb, a fluorescent bulb, a halogen bulb, a xenon bulb, mercury-vapor lamp, a light-emitting diode (LED), or a laser or other coherent light source.

15. The device of claim 1, wherein the light source is connected to a power source.

16. The device of claim 15, wherein the power source comprises a battery.

17. The device of claim 15, wherein the power source comprises an inductive power source, a kinetic power generator, an inductive power source, or a solar or photovoltaic power source.

18. The device of claim 1, further comprising a visible power indicator light.

19. The device of claim 1, wherein the UV light source operates continuously.

20. The device of claim 1, wherein the UV light source emits timed pulses of UV light.

21. The device of claim 1, wherein the UV light source is motion-activated, use-activated, touch-activated, or activated by a learning or other determinant algorithm.

22. A device that incorporates, utilizes, uses, or applies a method of disinfecting a surface of an object, whereby at least a part of the object including the surface is manufactured from UV light transmissive (translucent or transparent) material, and the UV light source(s) is/(are) incorporated behind, within, beneath, or inside the object, and UV light is continuously or periodically applied to the surface from behind, within, beneath, or inside to disinfect the surface.

23. The device of claim 1, further comprising a UV light switch configured to activate the UV light, wherein operation of the door opening means also activates the UV light.
